(19) United States
(12) Patent Application Publication
(10) Pub. No.: US 2023/0266204 A1
(43) Pub. Date: Aug. 24, 2023

(54) DRIED BLOOD SAMPLE COLLECTION DEVICE

(71) Applicant: True Diagnostics, Carlsbad, CA (US)

(72) Inventors: Jerry Lee, Carlsbad, CA (US); Brady Baehring, Carlsbad, CA (US)

(73) Assignee: True Diagnostics, Carlsbad, CA (US)

(21) Appl. No.: 17/677,811

(22) Filed: Feb. 22, 2022

(51) Int. Cl. G01N 1/08

(52) U.S. Cl. CPC G01N 1/08

(57) ABSTRACT

An apparatus and methods are provided for a dried blood sample collection device that facilitates self-testing by untrained personnel such as patients taking samples at home. The blood sample collection device comprises an actuator disposed through a distal barrel and a proximal barrel of the pen. Multiple frits for receiving blood samples are disposed within a distal end of the actuator. The frits are retained in a retracted position within the distal barrel by a biasing mechanism. A push button facilitates compressing the biasing mechanism and extending the frits through a distal opening of the distal barrel. The frits are configured to intake blood samples and store the samples in a dried state. A suitable desiccant may be used to cause the dried state. The actuator is configured to be removed from the blood sample collection device to enable retrieving dried blood samples from the distal end of the actuator.

### FIELD

Embodiments of the present disclosure generally relate to the collection of blood samples. More specifically, embodiments of the disclosure relate to an apparatus and methods for a dried blood sample collection device that facilitates self-testing by untrained personnel such as patients taking samples at home.

### BACKGROUND

Diagnostic blood samples conventionally are obtained from patients by medical professionals using a hypodermic syringe or a temporary cannula to draw off a succession of discrete samples into separate vials. More recently, dried blood spot samples have become a popular approach to obtaining diagnostic blood samples. Dried blood samples generally are collected by pricking a finger, heel, or toe with a lancet, spotting blood drops onto preprinted circles on specifically manufactured paper, such as an assay card, and then allowing the blood samples to dry in an open space. Dried blood samples advantageously enable blood samples to be collected by relatively untrained personnel or even by patients themselves. For example, untrained individuals can perform blood self-testing of their own blood sugar levels by using finger-prick blood spot kits.

A drawback to current dried blood samples, however, is that the samples generally must be dried for a minimum of 2-3 hours at room temperature. Further, the dried blood samples must be protected from humidity and moisture. Another drawback is that care must be taken not to contaminate the assay card or apply too much blood to the card, causing overlapping of samples on the card. Therefore, there is a need for an improved method and apparatus for use in blood sampling that reduces or eliminates one or more of the above drawbacks.

### SUMMARY

An apparatus and methods are provided for a dried blood sample collection device that facilitates self-testing by untrained personnel such as patients taking samples at home. The blood sample collection device comprises an actuator that is longitudinally disposed through a distal barrel and a proximal barrel of the pen. Multiple frits for receiving blood samples are disposed within a distal end of the actuator. The frits are retained in a retracted position within the distal barrel by a biasing mechanism. A push button coupled with the actuator facilitates compressing the biasing mechanism and extending the frits through a distal opening of the distal barrel. The frits are configured to intake blood samples and store the samples in a dried state. A suitable desiccant may be used to cause the dried state. The actuator is configured to be removed from the blood sample collection device to enable retrieving dried blood samples from the distal end of the actuator.

In an exemplary embodiment, a blood sample collection device comprises: a distal barrel coupled with a proximal barrel; an actuator longitudinally disposed throughout the distal barrel and the proximal barrel; one or more frits disposed within a distal end of the actuator; a biasing mechanism for retaining the distal end within an interior of the distal barrel; and a push button coupled with a proximal end of the actuator for extending the one or more frits through a distal opening of the distal barrel.

In another exemplary embodiment, the distal barrel is coupled to the proximal barrel by way of a plurality of threads. In another exemplary embodiment, the biasing mechanism is disposed between an interior of the distal barrel and a circumferential lip comprising the actuator.

In another exemplary embodiment, the push button is configured to be pushed is a distal direction to extend the one or more frits through the distal opening. In another exemplary embodiment, the biasing mechanism is configured to withdraw the one or more fits through the distal opening when the push button is released.

In another exemplary embodiment, the one or more frits are each configured to intake a blood sample and store the blood sample in a dried state. In another exemplary embodiment, the one or more frits comprise four individual frits that are removably coupled with the distal end of the actuator. In another exemplary embodiment, any one or more of the one or more frits includes a desiccant for causing the dried state. In another exemplary embodiment, the collection device is coated with a desiccant to cause the dried state. In another exemplary embodiment, any one or more of the one or more frits is configured to hold a volume of blood that is greater than about 1.0 μL. In another exemplary embodiment, any one or more of the one or more frits is configured to store a volume of about 60 μL of blood in a dried state.

In another exemplary embodiment, the actuator is configured to be removed from the blood sample collection device to enable retrieving filled frits from the distal end of the actuator. In another exemplary embodiment, the actuator includes a tubular casing that houses an extractor. In another exemplary embodiment, a distal end of the extractor is configured to eject the filled frits from the actuator when a proximal end of the extractor is pressed in a distal direction.

In another exemplary embodiment, each of the one or more frits is accompanied by a window disposed in the distal end. In another exemplary embodiment, the window facilitates blood being drawn into an adjacent frit without leaking or spreading into neighboring frits.

In an exemplary embodiment, a method for collecting a blood sample comprises: obtaining a blood sample collection device that includes one or more frits; pressing a push button comprising the blood sample collection device to extend the one or more frits through a distal opening; placing the one or more frits in contact with blood; allowing the blood to flow into the one or more frits; releasing the push button to retract the one or more frits through the distal opening; allowing the blood to become dried in the one or more frits; and sending the blood sample collection device to testing personnel.

In another exemplary embodiment, placing includes pressing the blood sample collection device longitudinally against the blood to fill the one or more frits with blood. In another exemplary embodiment, placing includes pressing a side of a distal end of the blood sample collection device against the blood to fill any one of the one or more frits with blood. In another exemplary embodiment, pressing a side of the distal end includes allowing the blood to flow through a window comprising the distal end into the any one of the one or more frits.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 illustrates a side view of an exemplary embodiment of a dried blood sample collection device in a retracted configuration;

FIG. 2 illustrates a side view of an exemplary embodiment of a dried blood sample collection device in a depressed configuration suitable for taking blood samples;

FIG. 3 illustrates a side view of an exemplary embodiment of a dried blood sample collection device in a sample collection configuration after a first sample of blood has been taken;

FIG. 4 illustrates a side view of an exemplary embodiment of a dried blood sample collection device in a full sample configuration wherein all frits comprising the dried blood sample collection device are filled with dried blood samples;

FIG. 5 illustrates a side view of an exemplary embodiment of an actuator that is removed from a dried blood sample collection device; and

FIG. 6 illustrates a side view of an exemplary embodiment of an actuator with multiple dried blood samples removed from the actuator.

While the present disclosure is subject to various modifications and alternative forms, specific embodiments thereof have been shown by way of example in the drawings and will herein be described in detail. The dried blood sample collection device should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the dried blood sample collection device disclosed herein may be practiced without these specific details. In other instances, specific numeric references such as “first lancet,” may be made. However, the specific numeric reference should not be interpreted as a literal sequential order but rather interpreted that the “first lancet” is different than a “second lancet.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

In general, dried blood samples are collected by pricking a finger, heel, or toe with a lancet, spotting blood drops onto preprinted circles on a specifically manufactured assay card and then allowing the blood samples to dry. A drawback to current dried blood samples, however, is that the samples generally must be dried for a minimum of 2-3 hours at room temperature. Another drawback is that care must be taken not to contaminate the assay card or apply too much blood to the card, causing overlapping of samples on the card. Embodiments, presented herein provide a dried blood sample collection device that eliminates one or more of these drawbacks, thereby facilitating self-testing by untrained personnel such as patients taking samples at home.

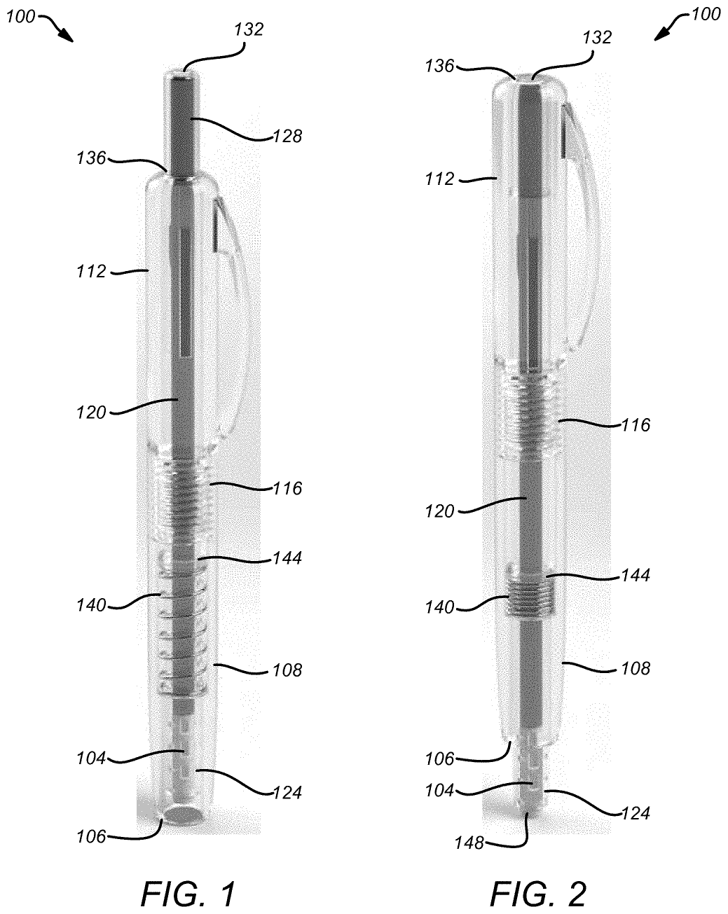

FIG. 1 illustrates a side view of an exemplary embodiment of a dried blood sample collection device 100 (hereinafter “collection device 100”) in a retracted configuration. The collection device 100 comprises a plurality of frits 104 that are retractable into a distal opening 106 of a distal barrel 108. The distal barrel 108 is coupled to a proximal barrel 112 by way of a plurality of threads 116. An actuator 120 is longitudinally disposed throughout the length of the distal and proximal barrels 108, 112. As shown in FIG. 1, the frits 104 are disposed within a distal end 124 of the actuator 120 while a proximal end 128 of the actuator 120 is disposed within a push button 132 that extends through a proximal opening 136 of the proximal barrel 112. A biasing mechanism 140 within the distal barrel 108 is disposed between an interior of the distal barrel 108 and a circumferential lip 144 comprising the actuator 120. The biasing mechanism 140 retains the collection device 100 in the retracted configuration wherein the frits 104 are withdrawn through the distal opening 106 into the distal barrel 108.

Although in the illustrated embodiment, the biasing mechanism 140 comprises a coil spring, it is contemplated that, in other embodiments, the biasing mechanism 140 may comprise any of various devices suitable for retaining the collection device 100 in the retracted configuration, without limitation. For example, in some embodiments, the biasing mechanism 140 may comprise a leaf spring made of shape memory metal. Further, in some embodiments, the biasing mechanism 140 may comprise a small piston that compresses air trapped inside a cylinder, such that when the trapped air expands the frits 104 are withdrawn through the distal opening 106 into the distal barrel 108.

The frits 104 are configured to intake blood samples and store the blood samples in a dried state for later testing by laboratory personnel. In some embodiments, the plurality of frits 104 comprises four individual frits 104 that are removably coupled with the distal end 124 of the actuator 120. It is contemplated that each of the frits 104 is configured to store a volume of about 60 microliters (μL) of blood in a dried state. In some embodiments, however, the frits 104 may be configured to hold a volume of blood that is greater than about 1.0 μL. For example, in some embodiments, the frits 104 may be configured to hold a volume of blood ranging between about 1.0 μL and about 90 μL. In some embodiments, the frits 104 may include any of various desiccants that are suitable for causing the blood samples to quickly dry within the frits 104. Further, in some embodiments, the collection device 100 may be coated or coupled with a suitable desiccant to cause blood samples within the frits 104 to assume a dried state. It is envisioned that, in some embodiments, the desiccant may cause the blood samples to assume the dried state within a time period ranging between about 30 mins and about 2 hours. Further, it is contemplated that the desiccant may comprise any one or more of montmorillonite clay, silica gel, indicating silica gel, molecular sieve, calcium oxide, calcium sulfate, any combination thereof, and the like, without limitation.

In operation, such as when a practitioner uses the collection device 100 to take a blood sample, the practitioner may begin by pressing the push button 132 into the proximal barrel 112 to cause the frits 104 to extend through the distal opening 106 of the distal barrel 108. For example, FIG. 2 illustrates a side view of the collection device 100 of FIG. 1 in a depressed configuration suitable for taking a blood sample. In the depressed configuration, the push button 132 is pushed into the proximal opening 136 of the proximal barrel 112, thereby compressing the biasing mechanism 140 and extending a distal frit 148 through the distal opening 106 of the distal barrel 108. Once exposed, the distal frit 148 may be placed in contact with a suitable source of blood to receive a blood sample. In some embodiments, the distal frit 148 may include an incorporated lancet, or other suitable device, for pricking a subject from whom the blood sample is to be taken. In some embodiments, however, a separate lancet may be used to cause a source of blood to flow before the collection device 100 is used to retrieve the blood sample.

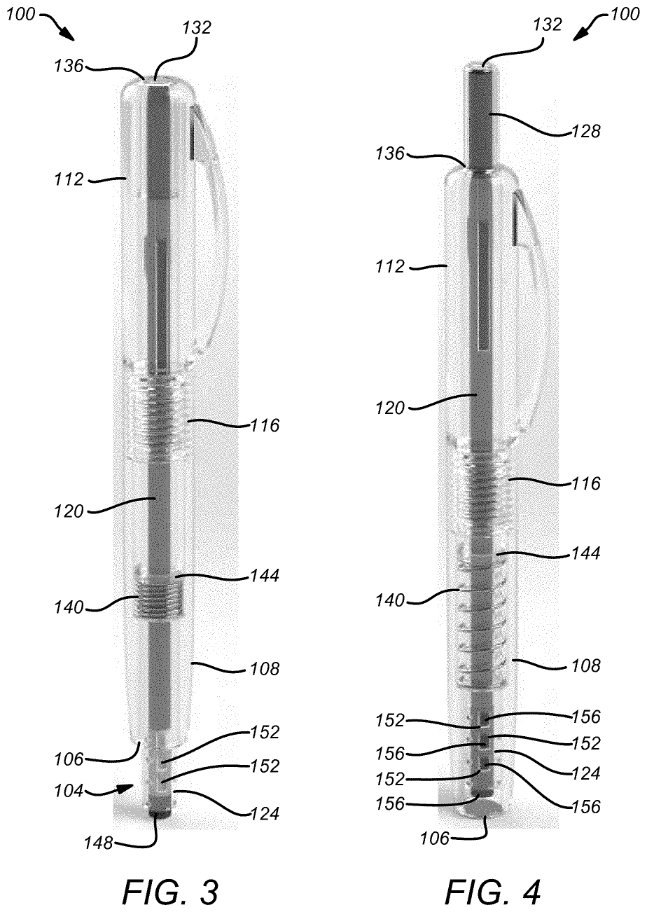

Moreover, the collection device 100 may be used to retrieve a relatively large blood sample that fills all the frits 104 at one time or a plurality of smaller blood samples that separately fills the frits 104. For example, FIG. 3 illustrates a side view of an exemplary embodiment of a collection device 100 in a sample collection configuration after a first sample of blood has been taken. As shown, the distal frit 148 is filled with an individual blood sample while the remaining frits 104 are left unfilled for future blood samples. As will be appreciated, the distal frit 148 may be filled with a blood sample by pressing the collection device 100 longitudinally into a blood source, such as pricked finger. It is contemplated, however, that the future blood samples may be taken by pressing the blood source against a side of the frits 104 to cause the blood to flow into each fit 104 to be filled. As shown in FIG. 3, each frit 104 is accompanied by a window 152 disposed in the distal end 124 whereby blood may be drawn into the fit 104 without leaking or spreading into neighboring frits 104.

Once the frits 104 have been desirably filled with blood samples, the collection device 100 may be placed into the retracted configuration and then sent to laboratory personnel for testing. For example, FIG. 4 illustrates a side view of an exemplary embodiment of a collection device 100 in a full sample configuration wherein all the frits 104 comprising the pen have been filled with sampled blood that is dried. As mentioned herein, the frits 104 may be filled with a single, relatively large blood sample that filled all the frits 104 at one time, or the frits 104 may contain a plurality of smaller, separately taken blood samples. As shown in FIG. 4, the collection device 100 includes multiple filled frits 156 that are retracted through the distal opening 106 into the distal barrel 108. As will be appreciated, when the practitioner releases the push button 132, the biasing mechanism 140 pushes the actuator 120 through the proximal opening 136 of the proximal barrel 112, thereby withdrawing the filled frits 156 into the distal barrel 108. It is contemplated that housing the filled frits 156 within the distal barrel 108 serves to protect the dried blood samples until the frits 156 are removed by laboratory personnel for testing.

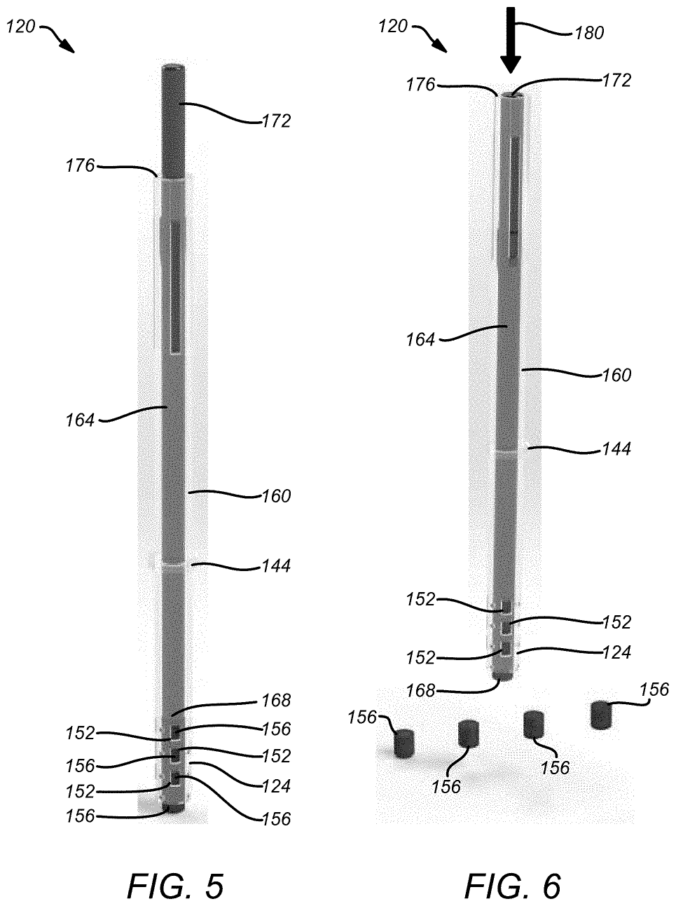

FIG. 5 illustrates a side view of an exemplary embodiment of an actuator 120 removed from the collection device 100. The actuator 120 may be removed from the collection device 100 by laboratory personnel during retrieving filled frits 156 at a distal end 124 of the actuator 120. As shown in FIG. 5, the actuator 120 includes a tubular casing 160 that houses an extractor 164. A distal end 168 of the extractor 164 is adjacent to the filled frits 156 while a proximal end 172 of the extractor 164 extends beyond a proximal edge 176 of the tubular casing 160. The proximal edge 176 is configured to receive the push button 132 (see FIG. 1), such that depressing the push button 132 does not prematurely eject the frits 104 during taking blood samples, as discussed with respect to FIG. 3.

Once the push button 132 is removed, as shown in FIGS. 5-6, the proximal end 172 of the extractor 164 may be pressed in a distal direction 180 to eject the filled frits 156 out of the distal end 124 of the tubular casing 160. In particular, pressing the extractor 164 in the distal direction 180 causes the distal end 168 of the extractor 164 to push the filled frits 156 in the distal direction 180. As such, laboratory personnel may use the extractor 164 to remove and separate the filled frits 156, as shown in FIG. 6.

It is contemplated that the collection device 100 is suitable for human blood sampling. In particular, the collection device 100 is suitable for patient's desiring to self-test in a home environment and then mail the collection device 100 to a suitable laboratory for testing. It is contemplated while the collection device 100 is configured to store whole blood samples, in some embodiments, the collection device 100 may be configured for collecting and storing blood serum samples, without limitation. Further, it should be understood that the collection device 100 is not limited to human blood sampling, but rather the collection device 100 may be used for animal blood sampling, without limitation. For example, the collection device 100 may be used by dog or cat owners desiring to perform blood sampling at home before sending the dried samples to a veterinary laboratory. To this end, the collection device 100 may be used to withdraw blood samples from the ears of dogs and cats for easy blood sampling. The collection device 100 may be put in the retracted configuration, discussed herein, and then mailed to a veterinary laboratory.

While the dried blood sample collection device has been described in terms of particular variations and illustrative figures, those of ordinary skill in the art will recognize that the dried blood sample collection device is not limited to the variations or figures described. In addition, where methods and steps described above indicate certain events occurring in certain order, those of ordinary skill in the art will recognize that the ordering of certain steps may be modified and that such modifications are in accordance with the variations of the dried blood sample collection device. Additionally, certain of the steps may be performed concurrently in a parallel process when possible, as well as performed sequentially as described above. To the extent there are variations of the dried blood sample collection device, which are within the spirit of the disclosure or equivalent to the dried blood sample collection device found in the claims, it is the intent that this patent will cover those variations as well. Therefore, the present disclosure is to be understood as not limited by the specific embodiments described herein, but only by scope of the appended claims.

What is claimed is:

1. A blood sample collection device, comprising:
a distal barrel coupled with a proximal barrel;
an actuator longitudinally disposed throughout the distal barrel and the proximal barrel;
one or more frits disposed within a distal end of the actuator;
a biasing mechanism for retaining the distal end within an interior of the distal barrel; and
a push button coupled with a proximal end of the actuator for extending the one or more frits through a distal opening of the distal barrel.

2. The blood sample collection device of claim 1, wherein the distal barrel is coupled to the proximal barrel by way of a plurality of threads.

3. The blood sample collection device of claim 1, wherein the biasing mechanism is disposed between an interior of the distal barrel and a circumferential lip comprising the actuator.

4. The blood sample collection device of claim 1, wherein the push button is configured to be pushed is a distal direction to extend the one or more frits through the distal opening.

5. The blood sample collection device of claim 4, wherein the biasing mechanism is configured to withdraw the one or more frits through the distal opening when the push button is released.

6. The blood sample collection device of claim 1, wherein the one or more frits are each configured to intake a blood sample and store the blood sample in a dried state.

7. The blood sample collection device of claim 6, wherein the one or more frits comprise four individual frits that are removably coupled with the distal end of the actuator.

8. The blood sample collection device of claim 7, wherein any one or more of the one or more frits includes a desiccant for causing the dried state.

9. The blood sample collection device of claim 7, wherein the collection device is coated with a desiccant to cause the dried state.

10. The blood sample collection device of claim 6, wherein any one or more of the one or more frits is configured to hold a volume of blood that is greater than about 1.0 μL.

11. The blood sample collection device of claim 10, wherein any one or more of the one or more frits is configured to store a volume of about 60 μL, of blood in a dried state.

12. The blood sample collection device of claim 1, wherein the actuator is configured to be removed from the blood sample collection device to enable retrieving filled frits from the distal end of the actuator.

13. The blood sample collection device of claim 1, wherein the actuator includes a tubular casing that houses an extractor.

14. The blood sample collection device of claim 13, wherein a distal end of the extractor is configured to eject the filled frits from the actuator when a proximal end of the extractor is pressed in a distal direction.

15. The blood sample collection device of claim 1, wherein each of the one or more frits is accompanied by a window disposed in the distal end.

16. The blood sample collection device of claim 15, wherein the window facilitates blood being drawn into an adjacent fit without leaking or spreading into neighboring frits.

17. A method for collecting a blood sample, comprising:
obtaining a blood sample collection device that includes one or more frits;
pressing a push button comprising the blood sample collection device to extend the one or more frits through a distal opening;
placing the one or more frits in contact with blood;
allowing the blood to flow into the one or more frits;
releasing the push button to retract the one or more frits through the distal opening; and
allowing the blood to become dried in the one or more frits.

18. The method of claim 17, wherein placing includes pressing the blood sample collection device longitudinally against the blood to fill the one or more frits with blood.

19. The method of claim 17, wherein placing includes pressing a side of a distal end of the blood sample collection device against the blood to fill any one of the one or more frits with blood.

20. The method of claim 19, wherein pressing a side of the distal end includes allowing the blood to flow through a window comprising the distal end into the any one of the one or more frits.
